(19) United States
(12) Patent Application Publication
(10) Pub. No.: US 2011/0060169 A1
(43) Pub. Date: Mar. 10, 2011

(54) HYDROGENATION CATALYST AND PROCESS FOR PREPARING ALCOHOLS BY HYDROGENATION OF CARBONYL COMPOUNDS

(75) Inventors: Alfred Kaizik, Marl (DE); Thomas Quandt, Marl (DE); Hans-Gerd Lueken, Marl (DE); Wilfried Bueschken, Haltern am See (DE)

(73) Assignee: Evonik Oxeno GmbH, Marl (DE)

(21) Appl. No.: 12/674,910

(22) PCT Filed: Jul. 7, 2008
(86) PCT No.: PCT/EP2008/058780

§ 371 Date: Feb. 24, 2010

(30) Foreign Application Priority Data
Aug. 31, 2007 (DE) 102007041380.9

(51) Int. Cl. C07C 29/141; B01J 21/06; B01J 21/04; B01J 37/08

(52) U.S. Cl. 568/881; 502/244; 502/250; 502/306; 502/328; 502/340; 502/245

(57) ABSTRACT

The invention relates to a hydrogenation catalyst which comprises a support material and at least one hydrogenation-active metal and in which the support material is based on titanium dioxide, zirconium dioxide, aluminium oxide, silicon oxide or mixed oxides thereof and the hydrogenation-active metal is at least one element from the group consisting of copper, cobalt, nickel, chromium, wherein the support material contains the element barium.

The invention further relates to a process for preparing alcohols by hydrogenation of carbonyl compounds, in which the hydrogenation is carried out in the presence of such a hydrogenation catalyst.

The invention relates to a hydrogenation catalyst and a process for preparing alcohols by hydrogenation of carbonyl compounds, in particular aldehydes.

Alcohols can be prepared by catalytic hydrogenation of carbonyl compounds which have been obtained, for example, by hydroformylation of olefins. Large quantities of alcohols are used as solvents and as intermediates for preparing many organic compounds. Important downstream products of alcohols are plasticizers and detergents.

It is known that carbonyl compounds, in particular aldehydes, can be catalytically reduced by means of hydrogen to form alcohols. Use is frequently made of catalysts which contain at least one metal of groups 1 b, 2b, 6b, 7b and/or 8 of the Periodic Table of the Elements. The hydrogenation of aldehydes can be carried out continuously or batchwise in the gas phase or liquid phase using pulverulent or pelletized catalysts.

The industrial preparation of alcohols by hydrogenation of aldehydes from the oxo process (hydroformylation of olefins) is, especially in the case of large-volume products, carried out in the gas phase or liquid phase using fixed-bed catalysts in continuous processes.

Compared to gas-phase hydrogenation, liquid-phase hydrogenation has the more favourable energy balance and the higher space-time yield. As the molar mass of the aldehyde to be hydrogenated increases, i.e. as the boiling point increases, the advantage of the more favourable energy balance increases. Higher aldehydes having more than 7 carbon atoms are therefore preferably hydrogenated in the liquid phase.

However, hydrogenation in the liquid phase has the disadvantage that the formation of high boilers by subsequent and secondary reactions is favoured because of the high concentrations both of aldehydes and of alcohols. Thus, aldehydes can more readily undergo aldol reactions (addition and/or condensation) and form hemiacetals or full acetals with alcohols. The acetals formed can undergo elimination of water or alcohol to form enol ethers which are hydrogenated under the reaction conditions to form the saturated ethers. These secondary by-products thus reduce the yield. The by-products referred to as high boilers can at best be partly redissociated into products of value such as starting aldehydes and product alcohols in downstream plants.

Industrial aldehyde mixtures used for hydrogenation frequently already contain high boilers in various concentrations.

The hydroformylation of olefins in the presence of cobalt catalysts gives crude aldehydes which contain not only formates but also aldol products, higher esters and ethers and also acetals as high boilers. If these mixtures are hydrogenated in the gas phase, the major part of the high boilers can be separated off in the vaporizer and worked up to give products of value in a separate process step.

On the other hand, in liquid-phase hydrogenation the high boilers remain in the reactor feed. They are mostly hydrogenated in the hydrogenation stage, so that it is no longer possible to recover a product of value from them.

In U.S. Pat. No. 5,059,710, the yield of alcohols by hydrogenation of crude aldehydes is increased by redissociating part of the high boilers by means of water at elevated temperature to form aldehydes or alcohols in a process step preceding the hydrogenation. Hydrolysis and hydrogenation are therefore separate process stages; nothing is said about the water content of the mixture to be hydrogenated.

A similar process is disclosed in U.S. Pat. No. 4,401,834. Here too, dissociation of high boilers in the presence of water is carried out before the actual hydrogenation step.

GB 2 142 010 describes a process for hydrogenating crude aldehydes which have from 6 to 20 carbon atoms and contain high boilers and small amounts of sulphur compounds to the corresponding saturated alcohols. The hydrogenation is carried out in two reactors connected in series. The first reactor contains an MoS₂/C catalyst and the second reactor contains an Ni/Al₂O₃ catalyst. The hydrogenation in both reactors is carried out with addition of up to 10% of steam, based on the feedstream, in the temperature range from 180 to 260° C. and a hydrogen partial pressure of from 150 to 210 bar using a large excess of hydrogen. According to the examples, this is so large that the water added is present virtually only in the gas phase. The objective of this process is to suppress the formation of hydrocarbons by hydrogenolysis of the alcohols. Nothing is said about an increase or decrease in high boilers and formates in the hydrogenation.

U.S. Pat. No. 2,809,220 describes a liquid-phase hydrogenation of hydroformylation mixtures in the presence of water. Sulphur-containing catalysts are used as catalyst. The hydrogenation is carried out in the pressure range from 105 to 315 bar and the temperature range from 204 to 315° C. in the presence of from 1 to 10% of water, based on starting material. To keep the added water in the gas phase, a large excess of hydrogen (from 892 to 3566 standard m³ of hydrogen per m³ of starting material) is used. As regards the high excess of hydrogen, reference may be made to the discussion of GB 2 142 010. The high specific energy consumption continues to be a disadvantage of this process.

A further process for the hydrogenation of hydroformylation mixtures is disclosed in DE 198 42 370. Here, a description of how hydroformylation mixtures can be hydrogenated in the liquid phase over supported catalysts containing copper, nickel and chromium is given. Depending on the process by which the hydroformylation mixtures have been prepared (rhodium or cobalt processes), these mixtures contain water. The process disclosed is designed for the selective hydrogenation of aldehydes to alcohols without hydrogenation of the olefins which have not been reacted in the hydroformylation, i.e. the high boilers (especially acetals) are not converted into product of value. This is economically unfavourable and therefore capable of improvement.

DE 100 62 448 describes a process for the continuous hydrogenation of reaction mixtures from the hydroformylation of olefins having from 4 to 16 carbon atoms in the homogeneous liquid phase over fixed-bed catalysts which contain at least one element of the eighth transition group of the Periodic Table, with the homogeneous liquid phase of the output from the reactor still containing from 0.05 to 10% by mass of water and, in the steady state of the process, from 3 to 50% more hydrogen than is consumed by the hydrogenation being fed in. This process has the advantage that the proportion of high boilers in the output from the hydrogenation at the beginning of a hydrogenation period with fresh catalyst is very low. However, the proportion of high boilers increases and the yield of alcohols drops as the period of operation increases.

In WO 01/87809, the feed to the hydrogenation reactor is admixed with an amount of a salt-like base (M⁺) (Aⁿ⁻), where (M⁺) is an alkali metal ion or an equivalent of an alkaline earth metal ion and (Aⁿ⁻) is an anion of an acid having a pKa of greater than 2 and n is the valence of the anion, which is soluble therein in order to reduce the formation of by-products in the hydrogenation of aldehydes: A disadvantage of this process is that the salts added are present in the output from the hydrogenation after the hydrogenation. These materials can only be separated off with difficulty and thus incur costs. In the work-up by direct distillation, the salts added can be deposited in the distillation plant and lead to production malfunctions. If this does not occur, the salts added go into the distillation bottoms. These are then unusable for many purposes because of their salt content.

It is therefore an object of the invention to discover a hydrogenation catalyst and develop a hydrogenation process by means of which carbonyl compounds, in particular aldehydes, are hydrogenated highly selectively to the corresponding saturated alcohols, with the selectivity being kept virtually constant over a long period of time and the addition of materials which are difficult to remove from the hydrogenation product being unnecessary.

It has now been found that carbonyl compounds, in particular aldehydes, can be hydrogenated over a long period of time with high, virtually constant selectivity to the corresponding alcohols when a hydrogenation catalyst which comprises a support material and at least one hydrogenation-active metal and in which the support material is based on titanium dioxide, zirconium dioxide, aluminium oxide, silicon oxide or mixed oxides thereof and the hydrogenation-active metal is at least one element of the group consisting of copper, cobalt, nickel, chromium and the support material contains the element barium is used.

The invention accordingly provides a hydrogenation catalyst which comprises a support material and at least one hydrogenation-active metal and in which the support material is based on titanium dioxide, zirconium dioxide, aluminium oxide, silicon oxide or mixed oxides thereof and the hydrogenation-active metal is at least one element from the group consisting of copper, cobalt, nickel, chromium, characterized in that the support material contains the element barium.

The invention further provides a process for preparing alcohols by hydrogenation of carbonyl compounds, in which the hydrogenation is carried out in the presence of a hydrogenation catalyst as characterized above.

The process of the invention using the catalyst of the invention has a series of unexpected advantages.

The formation of high boilers in the hydrogenation of aldehydes in the liquid phase as a result of secondary reactions such as aldol addition, aldol condensation, acetal formation or ether formation is very low when the catalysts of the invention are used. The selectivity for alcohol formation remains virtually constant over a long period of time, typically more than 2000 hours of operation. The activity of the catalyst decreases only slowly. For example, in the hydrogenation of appropriate hydroformylation mixtures to form isononanol, the activity after about 2000 hours of operation is still more than 99% of the value after 50 hours (proportion by weight of isononanol in the output from the hydrogenation). No materials which can get into the output from the hydrogenation are leached from the catalyst material during the hydrogenation. As a result, the work-up by distillation gives salt-free fractions which allow them to be utilized more readily. Owing to the high selectivity, the hydrogenation temperature can be increased without an appreciable increase in secondary reactions occurring. As a result, the space-time yield can be increased or can be kept constant as the catalyst activity falls off, which leads to a prolonged operating life of the catalyst. Increasing the hydrogenation temperature allows better utilization of the heat of hydrogenation.

The hydrogenation catalyst of the invention comprises a support material which is based on titanium dioxide, zirconium dioxide, aluminium oxide, silicon oxide or mixed oxides thereof and contains the element barium and a hydrogenation-active metal which is at least one element from the group consisting of copper, cobalt, nickel, chromium applied to this support material.

As support precursor, it is possible to use aluminium oxide, aluminosilicate, silicon dioxide, titanium dioxide, zirconium dioxide. A preferred support precursor is aluminium oxide, in particular γ-aluminium oxide.

The support material or the support precursor generally has pores. In the support material used, a distinction can be made between micropores (pore diameter less than 2 nm), mesopores (pore diameter from 2 to 50 nm) and macropores (pore diameter greater than 50 nm). The porosity of the support materials can be uniformly microporous, mesoporous or macroporous, but any combination of these pore size classes can also be present. Bimodal pore size distributions are frequently encountered.

The porosity of the support materials is typically such that the average pore diameter is from about 10 to 30 nm, the BET surface area is from about 80 to 300 m²/g and the pore volume (determined by means of the cyclohexane method) is from about 0.4 to 0.9 ml/g.

Such support materials or support precursors are known per se and are commercially available in many forms.

The support precursor is reacted with a barium compound to give the finished support. In the support material, the barium is present in the oxidation state 2 as metal oxide, as salt of the support precursor, as mixed oxide or, if appropriate, as another compound. The content of barium compound, calculated as barium oxide and based on the reduced catalyst, is in the range from 0.1 to 2% by mass, in particular in the range from 0.3 to 0.7% by mass.

At least one hydrogenation-active metal from the group consisting of copper, chromium, nickel, cobalt is applied to the support material which has been modified in this way with barium. The catalyst can contain one or more of the hydrogenation-active metals. The catalyst of the invention preferably contains the metals copper, chromium, nickel. The catalyst particularly preferably contains the combination of the three metals copper, chromium and nickel as hydrogenation-active metal.

The total content of hydrogenation-active metals is, based on the reduced catalyst, in the range from 1 to 40% by mass, in particular in the range from 5 to 25% by mass, calculated as metal.

The process for producing the hydrogenation catalyst of the invention is carried out by applying a solution containing a barium compound to a support material based on titanium dioxide, zirconium dioxide, aluminum oxide, silicon oxide or mixed oxides thereof, drying the support material which has been treated in this way and subsequently calcining it in a first stage and applying a solution containing at least one compound of the elements copper, cobalt, nickel, chromium to the support material which has been treated in this way, drying the support material which has been treated in this way and subsequently calcining it in a second stage.

In the first stage of the process, one or more barium compounds can be applied to the support precursor. This is preferably effected by spraying a solution onto the support precursor or impregnating the support precursor with a solution containing one or more barium compounds.

Suitable barium compounds are, for example, barium acetate, barium chloride (hydrate), barium hydroxide octahydrate, barium nitrate, barium chloride dehydrate.

A preferred compound is barium nitrate.

The barium compounds are preferably applied as aqueous solution.

The application of the barium compound can be carried out in one step or in a plurality of steps, with the solutions used in the individual steps being able to differ in terms of concentration and composition.

After application of the barium compound, the raw support material is predried in the temperature range from 80 to 120° C. in a stream of air. If the barium compound is applied in a plurality of steps, drying can be carried out after each step. After predrying, the raw support material is calcined in the temperature range from 400 to 650° C., in particular in the range from 420° C. to 550° C.

After calcination, one or more of the hydrogenation-active metals copper, chromium, nickel, cobalt are applied to the support material. The application is carried out in a manner analogous to that described for the application of the barium compound, namely by treating the support material with a solution of the appropriate metal compounds. Preference is given to using aqueous solutions of compounds of the hydrogenation-active metals.

To prepare these solutions, it is possible to use, for example, the following compounds:

copper formate, copper acetate, copper chloride, copper nitrate, copper sulphate, copper acetylacetonate and the corresponding aquo and ammine complexes of these compounds;
cobalt formate, cobalt acetate, cobalt chloride, cobalt nitrate, cobalt sulphate, cobalt acetylacetonate and aquo and ammine complexes derived therefrom;
nickel formate, nickel acetate, nickel acetylacetonate, nickel chloride, nickel nitrate, nickel sulphate and aquo and ammine complexes derived therefrom;
chromium formate, chromium acetate, chromium acetylacetonate, chromium chloride; chromium nitrate, chromium sulphate and aquo and ammine complexes derived therefrom and also
ammonium chromate and ammonium dichromate.

If the catalyst of the invention is to contain more than one hydrogenation-active metal, the support is advantageously treated with a joint solution of compounds of the metals to be combined. However, it is also possible to apply appropriate solutions of the metals to be combined to the support in succession, with drying being able to be carried out after each step.

In a particularly preferred embodiment, the catalyst support is treated with a joint solution of compounds of the three metals copper, chromium and nickel.

After application of the compounds of the hydrogenation-active metals and predrying, the raw catalyst is calcined in the temperature range from 400° C. to 650° C., in particular in the temperature range from 420° C. to 550° C.

If the hydrogenation-active metals are applied as formates or nitrates to the support, it may be possible to omit calcination.

The catalysts of the invention are advantageously produced in a form which offers a low flow resistance during the hydrogenation, for example pellets, cylinders, extrudates or rings. In the production of the catalyst, the support precursor is usually brought into the appropriate form. Shaped support precursor is also commercially available.

The process of the invention for preparing alcohols by hydrogenation of carbonyl compounds is carried out in a manner known per se, but the hydrogenation is carried out in the presence of a hydrogenation catalyst according to the invention as described above.

In the process of the invention, the hydrogenation can be carried out continuously or batchwise over suspended finely divided or shaped, fixed-bed catalysts. Continuous hydrogenation over a fixed-bed catalyst, in which the product/starting material phase is mainly in the liquid state under the reaction conditions, is preferred.

If the hydrogenation is carried out continuously over a fixed-bed catalyst, it is advantageous to convert the catalyst into the active form before the hydrogenation. This can be effected by reduction of the catalyst by means of hydrogen-containing gases according to a temperature programme. The reduction can, if appropriate, be carried out in the presence of a liquid phase which is passed over the catalyst, as described, for instance, in DE 199 33 348.

The process of the invention is carried out in cocurrent in the trickle phase or preferably in the liquid phase in three-phase reactors, with the hydrogen being finely dispersed in a known manner in the liquid feed/product stream. In the interests of uniform distribution of liquid, improved removal of the heat of reaction and a high space-time yield, the reactors are preferably operated at high liquid space velocities of from 15 to 120 m³, in particular from 25 to 80 m³, per m² of cross section of the empty reactor and hour. If a reactor is operated isothermally and in a single pass, the specific space velocity over the catalyst (LHSV) can be from 0.1 to 10 h⁻¹.

The process of the invention is carried out using hydrogen in a pressure range from 5 to 100 bar, preferably from 5 to 40 bar, particularly preferably in the range from 10 to 25 bar. The hydrogenation temperatures are in the range from 120 to 220° C., in particular from 140 to 190° C.

The hydrogen used for the hydrogenation can contain inert gases such as methane or nitrogen. Preference is given to using hydrogen having a purity of greater than 98%, in particular greater than 99%.

Various process variants can be selected for the process of the invention. It can be carried out adiabatically or virtually isothermally, i.e. with a temperature rise of less than 10° C., in one or more stages. In the latter case, all reactors, advantageously tube reactors, can be operated adiabatically or virtually isothermally or one or more can be operated adiabatically and the others can be operated virtually isothermally. Furthermore, it is possible to hydrogenate the carbonyl compounds or mixtures of carbonyl compounds in the presence of water in a single pass or with product recirculation.

It is in principle possible to hydrogenate all carbonyl compounds to the corresponding alcohols using the hydrogenation catalyst of the invention and the process of the invention. In particular, it is possible to hydrogenate aldehydes to primary alcohols, ketones to secondary alcohols, α,β-unsaturated aldehydes to saturated primary alcohols and α,β-unsaturated ketones to saturated secondary alcohols. These carbonyl compounds can have further functional groups such as hydroxyl or alkoxy groups. Furthermore, further nonconjugated olefinic double bonds can be present and these can, depending on the catalyst and on the further process conditions, remain unhydrogenated or be partially or completely hydrogenated.

The hydrogenation of α,β-unsaturated ketones or aldehydes is preferably carried out without addition of water and the hydrogenation of nonconjugated ketones and aldehydes is preferably carried out with addition of water, as described, for example, in DE 100 62 448.

The process of the invention is preferably used to hydrogenate carbonyl compounds having from 4 to 25 carbon atoms, in particular saturated or unsaturated aldehydes or ketones having from 4 to 25 carbon atoms.

To minimize secondary reactions and thus increase the alcohol yield, it is advantageous to limit the concentration of carbonyl compounds in the feed to the reactor. Particularly in the hydrogenation of hydroformylation mixtures having from 8 to 17 carbon atoms, the aldehyde content in the feed to the reactor is from 1 to 35% by mass, in particular from 5 to 25% by mass. The desired concentration range can in the case of reactors which are operated in a recycle mode be set via the recycle ratio (ratio of recycled hydrogenation output to feed).

The carbonyl compounds used in the process of the invention can be prepared in various ways:

α,β-unsaturated ketones can, for example, be prepared by condensation of two ketones or condensation of a ketone with an aldehyde, for example oct-3-en-2-one from n-pentanal and acetone;
α,β-unsaturated aldehydes can be prepared by aldol condensation of aldehydes, for example 2-ethylhex-2-enal from n-butanal, 2-propylhept-2-enal from n-pentanal or a mixture of isomeric decenals by condensation of at least two different C₅-aldehydes. Preference is given to using a decenal mixture prepared by condensation of C₅-aldehydes, in particular valeraldehyde.

The nonconjugated unsaturated aldehydes used in the process of the invention are predominantly prepared by hydroformylation.

The starting materials for preparing the aldehydes or the reaction mixture by hydroformylation are olefins or mixtures of olefins having from 3 to 24, in particular from 4 to 16, carbon atoms and terminal or internal C—C double bonds, e.g. 1-butene, 2-butene, isobutene, 1- or 2-pentene, 2-methyl-1-butene, 2-methyl-2-butene, 3-methyl-1-butene, 1-, 2- or 3-hexene, the C₆-olefin mixture obtained in the dimerization of propene (dipropene), heptenes, 2- or 3-methyl-1-hexene, octenes, 2-methylheptenes, 3-methylheptenes, 5-methyl-2-heptene, 6-methyl-2-heptene, 2-ethyl-1-hexene, the mixture of isomeric C₈-olefins obtained in the dimerization of butenes (dibutene), nonenes, 2- or 3-methyl-octenes, the C₉-olefin mixture obtained in the trimerization of propene (tripropene), decenes, 2-ethyl-1-octene, dodecenes, the C₁₂-olefin mixture obtained in the tetramerization of propene or the trimerization of butenes (tetrapropene or tributene), tetradecenes, pentadecenes, hexadecenes, the C₁₆-olefin mixture obtained in the tetramerization of butenes (tetrabutene) and olefin mixtures prepared by cooligomerization of olefins having different numbers of carbon atoms (preferably from 2 to 4), if appropriate after separation by distillation into fractions having the same or similar chain length(s). It is likewise possible to use olefins or olefin mixtures which have been produced by Fischer-Tropsch synthesis and olefins which have been obtained by oligomerization of ethene or olefins which can be obtained by means of metathesis reactions. Preferred starting materials for the preparation of the hydroformylation mixtures are C₈-, C₉-, C₁₂-, C₁₅- or C₁₆-olefin mixtures. In the process of the invention, particular preference is given to using hydroformylation mixtures prepared from C₈- or C₁₂-olefins or C₈- or C₁₂-olefin mixtures. The C₉-aldehyde isononanal which can be obtained by hydroformylation of dibutene is particularly preferably used.

The olefins are hydroformylated in a customary fashion and then give the starting materials for the hydrogenation process of the invention. The hydroformylation is generally carried out using rhodium or cobalt catalysts, either with or without additives which stabilize the complex, e.g. organic phosphines or phosphites. The temperatures and pressures can, depending on the catalyst or olefin, vary within wide limits. A description of the hydroformylation of olefins may be found, for example, in J. Falbe, New Syntheses with Carbon Monoxide, Springer-Verlag, Heidelberg-New York, 1980, page 99 ff., and in Kirk-Othmer, Encyclopedia of Chemical Technology, Volume 17, 4th edition, John Wiley & Sons, pages 902 to 919 (1996).

The reaction mixtures from the hydroformylation are advantageously firstly freed of the catalyst before use in the process of the invention. If a cobalt catalyst has been used, this can be effected by depressurization, oxidation of the cobalt carbonyl compounds remaining in the hydroformylation mixture in the presence of water or aqueous acid and removal of the aqueous phase. Cobalt removing processes are well known; see, for example, J. Falbe, loc. cit., Kirk-Othmer, loc. cit., 164, 175, BASF process.

If a rhodium compound is used as hydroformylation catalyst, it can, for example, be separated off as distillation residue by means of thin film evaporation.

The reaction mixtures from the cobalt-catalysed hydroformylation which have been freed of the hydroformylation catalyst generally contain from 3 to 40% by mass, usually from 5 to 30% by mass, of low boilers, mainly unreacted olefins, together with the corresponding saturated hydrocarbons and from 0.05 to 5% by mass of water, from 30 to 90% by mass of aldehydes, from 5 to 60% by mass of alcohols, up to 10% by mass of formates of these alcohols and from 3 to 15% by mass of high boilers.

However, it should be emphasized that the process of the invention can also be carried out using hydroformylation mixtures whose composition in this or any respect does not correspond to the above. Thus, for example, the hydrocarbons (olefins and paraffins) can be separated off from the hydroformylation mixture prior to the hydrogenation.

The hydrogenation outputs obtained by the process of the invention are worked up by distillation. This is carried out at atmospheric pressure or under reduced pressure. In the case of high-boiling alcohols, distillation under reduced pressure is preferred.

The following examples serve to illustrate the invention without the invention being restricted thereto.

### EXAMPLE 1

### Production of a Hydrogenation Catalyst (not According to the Invention)

A commercial aluminium oxide support (from Axens) in the form of extrudates having a diameter of about 1.2 mm, a BET surface area of about 260 m²/g and a pore volume (determined by means of the cyclohexane method) of 0.7 ml/g was firstly modified by partial neutralization of acid sites by means of sodium compounds. For this purpose, 500 g of the extrudates were placed in a glass tube and this was evacuated for about 30 minutes. The impregnation solution, viz. a dilute aqueous sodium hydroxide solution (w(NaOH)=0.24%), was subsequently drawn up from the bottom to above the upper surface of the bed of solid. After a residence time of about 15 minutes, the solution which had not been taken up by the support was drained. The impregnated extrudates were firstly dried at 120° C. in a stream of air, subsequently heated at 2 K/min to 450° C. and calcined at this temperature for 6 hours. The catalyst precursor produced in this way formally contained 0.1% by mass of sodium. The sodium-modified aluminium oxide support was subsequently impregnated by vacuum impregnation with an ammoniacal solution containing nickel, copper and chromium compounds. For this purpose, an ammonium dichromate solution (calculated chromium content: 7.1% by mass) was firstly stirred into a mixture of tetramminecopper carbonate solution (Cu content by electrogravometric determination: 13.9% by mass, NH₃ content by the Kjeldahl method: 13.0% by mass, density at 20° C.: 1.242 g/cm³) and hexamminenickel carbonate solution (Ni content calculated from starting compound: 11.2% by mass, NH₃ content by the Kjeldahl method: 18.6% by mass, density at 20° C.: 1.29 g/cm³). The content of copper, nickel and chromium of the dark green impregnation solution calculated from the starting compounds was 8.1% by mass of copper, 3.6% by mass of nickel and 0.7% by mass of chromium. The density of the solution was 1.26 g/cm³. For vacuum impregnation, 500 g of the extrudates were placed in a glass tube and this was evacuated for about 30 minutes. The impregnation solution was subsequently drawn up from the bottom to above the upper surface of the bed of solid. After a residence time of about 15 minutes, the solution which had not been taken up by the support was drained. The moist pellets were firstly dried at 120° C. in a stream of air, subsequently heated at 3 K/min to 450° C. and calcined at this temperature for 10 hours. After the calcination, the catalyst formally contained: 86% by mass of Al₂O₃, 6.4% by mass of Cu, 2.9% by mass of Ni, 0.6% by mass of Cr and 0.09% by mass of Na.

### EXAMPLE 2

### Production of a Hydrogenation Catalyst According to the Invention

A commercial aluminium oxide support (from Axens) in the form of extrudates having a diameter of about 1.2 mm, a BET surface area of about 260 m²/g and a pore diameter (determined by means of the cyclohexane method) of 0.7 ml/g was firstly modified by partial neutralization of acid sites by means of barium compounds. For this purpose, 500 g of the extrudates were placed in a glass tube and this was evacuated for about 30 minutes. The impregnation solution, viz. a dilute aqueous barium nitrate solution (w(Ba)=0.4%), was subsequently drawn up from the bottom to above the upper surface of the bed of solid. After a residence time of about 15 minutes, the solution which had not been taken up by the support was drained. The impregnated extrudates were firstly dried at 120° C. in a stream of air, subsequently heated at 2 K/min to 450° C. and calcined at this temperature for 6 hours. The catalyst precursor produced in this way formally contained 0.32% by mass of barium.

The barium-modified aluminium oxide support was subsequently impregnated by vacuum impregnation with an ammoniacal solution containing nickel, copper and chromium compounds. For this purpose, an ammonium dichromate solution (calculated chromium content: 7.1% by mass) was firstly stirred into a mixture of tetramminecopper carbonate solution (Cu content by electrogravometric determination: 13.9% by mass, NH₃ content by the Kjeldahl method: 13.0% by mass, density at 20° C.: 1.29 g/cm³) and hexamminenickel carbonate solution (Ni content calculated from starting compound: 10.6% by mass, NH₃ content by the Kjeldahl method: 18.0% by mass, density at 20° C.: 1.21 g/cm³). The content of copper, nickel and chromium of the dark green impregnation solution calculated from the starting compounds was 7.7% by mass of copper, 3.5% by mass of nickel and 0.8% by mass of chromium. The density of the solution was 1.23 g/cm³. For vacuum impregnation, 500 g of the extrudates were placed in a glass tube and this was evacuated for about 30 minutes. The impregnation solution was subsequently drawn up from the bottom to above the upper surface of the bed of solid. After a residence time of about 15 minutes, the solution which had not been taken up by the support was drained. The moist pellets were firstly dried at 120° C. in a stream of air, subsequently heated at 3 K/min to 450° C. and calcined at this temperature for 10 hours. After the calcination, the catalyst formally contained: 87% by mass of Al₂O₃, 6.3% by mass of Cu, 2.8% by mass of Ni, 0.6% by mass of Cr and 0.3% by mass of Ba.

### EXAMPLE 3

### Hydrogenation of C₉-aldehyde in the Liquid Phase Over the Catalyst Produced in Example 1 (Comparison, not According to the Invention)

A reaction product mixture from the cobalt-catalysed hydroformylation of dibutene containing 60.65% by mass of the C₉-aldehyde isononanal was hydrogenated continuously in the liquid phase in a circulation apparatus at 180° C. and 25 bar absolute over 70.2 g (corresponding to 100 ml) of catalyst. 0.075 l/h of feed was fed in at a circulation rate of 45 l/h. The amount of offgas was 60 standard l/h. The analyses of the starting material and product are shown in Table 1. The analysis at time zero indicates the composition of the starting material.

TABLE 1

| Time of | C₈- | C₉-al | Formate | C₉-ol | |
|---|---|---|---|---|---|
| operation | hydrocarbons | (% by | (% by | (% by | High boilers |
| (hours) | (% by mass) | mass) | mass) | mass) | (% by mass) |
| 0 | 6.31 | 60.65 | 3.95 | 27.15 | 1.94 |
| 50 | 6.21 | 0.52 | 0.48 | 90.81 | 1.98 |
| 500 | 6.05 | 0.72 | 0.49 | 89.99 | 2.75 |
| 1000 | 5.98 | 1.04 | 0.49 | 88.80 | 3.68 |
| 1500 | 5.97 | 1.67 | 0.51 | 87.85 | 3.98 |
| 2000 | 5.81 | 1.99 | 0.51 | 87.12 | 4.60 |

As can be seen from Table 1, increasing formation of high boilers occurred as the period of operation increased in the hydrogenation of isononanal over the standard catalyst. The residual contents of C₉-aldehyde increased from 0.52% by mass at the beginning of the hydrogenation to about 2% by mass after 2000 hours of operation. The decrease in the catalyst activity and the formation of high boilers resulted in the yield of the desired product isononanol in the hydrogenation being reduced as the period of operation increased. The content of C₉-alcohol, which was about 90.8% by weight at the beginning of the hydrogenation, dropped to about 87.1% by weight over 2000 hours.

### EXAMPLE 4

### Hydrogenation of C₉-aldehyde Over the Catalyst Produced in Example 2 (According to the Invention)

A reaction product mixture from the cobalt-catalysed hydroformylation of dibutene containing 60.34% by mass of the C₉-aldehyde isononanal was hydrogenated continuously in the liquid phase in a circulation apparatus at 180° C. and 25 bar absolute over 69.5 g (corresponding to 99 ml) of catalyst. Long-term testing was carried out under reaction conditions comparable to those for the standard catalyst (from Example 1) in Example 3. 0.075 l/h of feed was fed in at a circulation rate of 45 l/h. The amount of offgas was 60 standard l/h. The analyses of the starting material and product are shown in Table 2.

TABLE 2

| Time of | C₈- | C₉-al | Formate | C₉-ol | |
|---|---|---|---|---|---|
| operation | hydrocarbons | (% by | (% by | (% by | High boilers |
| (hours) | (% by mass) | mass) | mass) | mass) | (% by mass) |
| 0 | 6.57 | 60.34 | 3.15 | 28.07 | 1.87 |
| 50 | 6.13 | 0.62 | 0.45 | 90.85 | 1.95 |
| 500 | 6.11 | 0.73 | 0.36 | 91.01 | 1.75 |
| 1000 | 6.02 | 0.78 | 0.31 | 91.02 | 1.86 |
| 1500 | 6.04 | 0.89 | 0.31 | 90.85 | 1.87 |
| 2000 | 6.03 | 1.02 | 0.28 | 90.65 | 2.03 |

As can be seen from Table 2, smaller amounts of high boilers were formed in the hydrogenation of crude isononanal over the BaO-modified Cu/Cr/Ni catalyst according to the invention (from Example 2) compared to the standard catalyst (from Example 1). The residual C₉-aldehyde contents increased significantly more slowly with time of operation than in the case of the standard catalyst, which indicates a smaller decrease in activity.

The improved selectivity and activity of the catalyst according to the invention compared to unmodified standard catalyst resulted in the yields of the desired product isononanal not being reduced appreciably during the period of operation. The high C₉-alcohol contents of over 90.5% by weight were maintained even after 2000 hours.

1. A hydrogenation catalyst which comprises a support material and at least one hydrogenation-active metal and in which the support material is based on titanium dioxide, zirconium dioxide, aluminium oxide, silicon oxide or mixed oxides thereof and the hydrogenation-active metal is at least one element from the group consisting of copper, cobalt, nickel, chromium, wherein the support material contains the element barium.

2. The hydrogenation catalyst according to claim 1, wherein the catalyst contains from 0.1 to 2% by mass of barium, calculated as barium oxide.

3. The hydrogenation catalyst according to claim 1, wherein the support material is based on aluminium oxide.

4. The hydrogenation catalyst according to claim 1, wherein it contains from 1 to 40% by mass of hydrogenation-active metals, calculated as metal.

5. The hydrogenation catalyst according to claim 1, wherein it contains the combination of the three metals copper, chromium and nickel as hydrogenation-active metal.

6. The hydrogenation catalyst according to claim 1, wherein it contains from 1 to 20% by mass of copper, from 0.2 to 6% by mass of chromium, from 1 to 20% by mass of nickel, in each case calculated as metal, and from 0.1 to 2% by mass of barium, calculated as metal oxide.

7. A process for producing a hydrogenation catalyst according to claim 1, wherein a solution containing a barium compound is applied to a support material based on titanium dioxide, zirconium dioxide, aluminium oxide, silicon oxide or mixed oxides thereof and the support material which has been treated in this way is dried and subsequently calcined in a first stage and a solution containing at least one compound of the elements copper, cobalt, nickel, chromium is applied to the support material which has been treated in this way and the support material which has been treated in this way is dried and subsequently calcined in a second stage.

8. The process according to claim 7, wherein the drying steps are carried out in the temperature range from 80 to 120° C. and calcinations steps are carried out in the temperature range from 400 to 650° C.

9. A process for preparing alcohols by hydrogenation of carbonyl compounds, wherein the hydrogenation is carried out in the presence of a hydrogenation catalyst according to claim 1.

10. The process according to claim 9, wherein the hydrogenation is carried out using hydrogen in a pressure range from 5 to 100 bar, and at a hydrogenation temperature of from 120 to 220° C.

11. The process according to claim 9, wherein saturated or unsaturated aldehydes or ketones having from 4 to 25 carbon atoms are used as carbonyl compounds.

12. The process according to claim 9, wherein carbonyl compounds obtained by hydroformylation are used.

13. The process according to claim 12, wherein hydroformylation mixtures prepared from C₈- or C₁₂-olefins or C₈- or C₁₂-olefin mixtures are used.

14. The process according to claim 12, wherein the C₉-aldehyde isononanal which can be obtained by hydroformylation of dibutene is used.

15. The process according to claim 9, wherein a decenal mixture prepared by condensation of C₅-aldehydes, is used.
